(19) United States
(12) Patent Application Publication
(10) Pub. No.: US 2006/0195172 A1
(43) Pub. Date: Aug. 31, 2006

(54) Multi-unit stent-graft

(75) Inventors: Qiyi Luo, Shanghai (CN); Shangdong Xu, Shanghai (CN); Honglin Nie, Shanghai (CN)

Correspondence Address: CROCKETT & CROCKETT, 24012 CALLE DE LA PLATA, SUITE 400, LAGUNA HILLS, CA, 92653, US

(73) Assignee: MicroPort Medical Co., Ltd.

(21) Appl. No.: 11/348,829

(22) Filed: Feb. 6, 2006

Related U.S. Application Data
(63) Continuation-in-part of application No. PCT/CN05/00815, filed on Aug. 17, 2004.

(30) Foreign Application Priority Data
Aug. 17, 2004 (CN) 200410053816.7

(51) Int. Cl. A61F 2/06

(52) U.S. Cl. 623/1.13

(57) ABSTRACT

A new stent-graft is provided with an improved structure design which comprises multiple stent-graft units with/without one or two open stents sutured to combine the uniform prosthesis without a connector bar in the place where flexing required. Each stent-graft unit comprises a stent sutured to a tubular graft. The new stent-graft can bend, provides a smooth inner surface and is stable after placement within an inner lumen of the human body.

This application is a continuation-in-part of International Application No. PCT/CN2005/000815, which claims priority to Chinese patent application CN 200420081915.1, filed Aug. 17, 2004.

### FIELD OF THE INVENTIONS

The inventions described below relate the field of bifurcated stent grafts and treatment of aortic aneurysms.

### BACKGROUND OF THE INVENTIONS

Aneurysms are localized, pathological, blood-filled dilatations of a blood vessel caused by a disease or weakening of the vessel's wall. Aneurysms occur more often in people over the age of fifty, but can occur in people of all age groups. A vessel in the body where aneurysms often occur is in the aorta, the main artery of the chest and abdomen. More than 15,000 Americans die each year due to ruptured aneurysms.

Surgical treatments for aneurysms have been performed for more than 50 years. During a typical surgical procedure for the treatment of an aneurysm, the diseased part of the aorta is replaced with a Dacron® or Teflon® graft that is carefully matched to the normal aorta and sewn in place by the surgeon. Although curative, this operation requires a large thoracic or abdominal incision, general anesthesia, and a hospital stay averaging 7-10 days for most patients. Even after surgery without complication, it often takes a month or two before patients can return to a full and normal life.

Less invasive treatments of aneurysms resulting from advances in catheter-based technologies have led to new treatments for aortic aneurysms. Now, endovascular grafting technology allows surgeons to repair the aneurysm in the ascending aorta, the aortic arch, descending thoracic aorta and abdominal aorta by delivering a graft through a small incision in the groin, rather than the traditional major open surgery.

A wide range of endovascular stent-grafts have been developed that are adapted for temporary or permanent implantation within a body lumen such as the abdominal aorta or thoracic aorta. Various types of stent-grafts provide uniquely beneficial support structure to modify the mechanics of the targeted vessel wall.

Present stent-grafts contain a longitudinal connecting bar connecting stent disposed on the proximal end of the stent-graft to stents disposed on the distal end of the stent-graft to maintain the total length of the stent-graft. While maintaining the total length of the stent-graft, use of a connector bar adversely affects flexibility of stent-grafts along the longitudinal axis. When a stent-graft having a connector bar is disposed in a curved artery, such as the aortic arch, the connector bar must be placed longitudinally along the outside curve of the arch of the aorta. Otherwise, if the connector bar is not placed along the outside curve of the arch, but instead is placed along the inside curve of the arch, the stent-graft can kink or buckle causing: a rough inner lumen to form in the stent-graft, a decrease in the contact surface between the graft and blood vessel, a decrease in the fixing performance of the stent-graft, higher blood flush force on the stent-graft and an increase in potential for stent-graft migration which can lead to injury or patient death. What is needed is a stent-graft without a connector bar for use in a curved artery where flexing is required for placement.

### SUMMARY

A multi-unit stent-graft and method of use is disclosed. The multi-unit stent-graft comprises multiple stent-graft units stacked upon each other, nested and sutured from graft to graft to adjacent stent-graft units with the proximal stent-graft unit overlapping partly the neighboring immediately adjacent stent-graft unit to combine to form a tubular stent without a connector bar. Each stent-graft unit comprises a stent coupled to a graft made of biocompatible material defining a tube. The multi-unit stent-graft may further comprise one or more open stent.

The multi-unit stent-graft may be provided as a uniform tubular stent-graft with or without an open-frame stent on one end or both ends. The multi-unit stent-graft may also be provided as a uniform bifurcated stent-graft comprising a trunk stent-graft portion connected with a first iliac branch stent-graft portion and connected to a branch port which in fluid communication with a second iliac branch stent-graft portion.

### BRIEF DESCRIPTION OF THE DRAWINGS

FIG. 1 is a multi-unit stent-graft with a proximal open stent.

FIG. 2 illustrates a stent-graft unit having a graft with a first end portion and a second end portion slightly exceeding the length of the stent.

FIG. 3 illustrates a stent-graft unit having a graft with a first end portion length slightly exceeding the length of the stent and a second end portion length extending beyond the stent by a greater length.

FIG. 4 illustrates a uniform bifurcated endoluminal prosthesis comprising a trunk stent-graft portion with a first iliac branch portion and a second iliac branch portion.

### DETAILED DESCRIPTION OF THE INVENTIONS

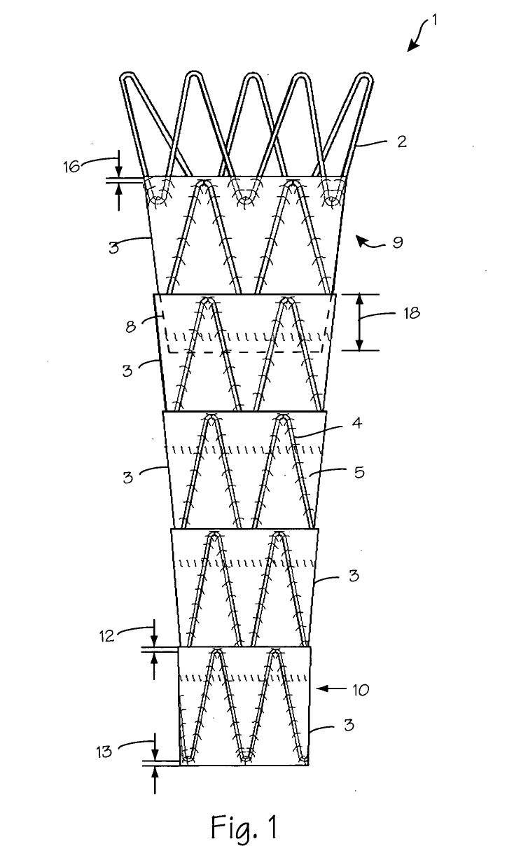

FIG. 1 shows a multi-unit stent-graft 1 with an open-frame stent 2 sutured on the proximal end of the proximal most stent-graft unit. The multi-unit stent-graft comprises two or more stent-graft units 3 stacked and nested within one another, sutured together and inter-connect to form a tubular endoluminal prosthesis. Each stent-graft unit comprises a supporting stent 4 sutured to a graft 5 made of biocompatible PET woven fabric defining a tube or having an inner diameter, outer diameter and a length in the longitudinal direction. The stent may be made of any suitable material, such as nitinol (in either pseudoelastic or shape memory form), CoCr alloy, or 316L/317L stainless steel, and may be balloon expandable or self-expanding. The stent 4 can be of zigzag shape structure, slotted tube or woven structure, and may be articulated as necessary to permit the overall stent-graft to conform to the anatomy of the patient. The stent 4 may be disposed within the inner diameter of the graft, disposed about the outer diameter of the graft or disposed within the matrix of the PET woven fabric.

The distal most stent-graft unit 10 comprises a graft 5 with a first end portion 12 slightly exceeding the length of the stent and a second end portion 13 slightly exceeding the length of the stent. The other stent-graft units 3 have graft 5 with the first end portion 16 slightly exceeding the length of the stent and the second end portion 18 exceeding the length of the stent to a greater extent, creating an overlapping length 8 in the stent-graft unit. The stent-graft units 3 are interconnected and sutured together from graft to graft to adjacent stent-graft units with the proximal stent-graft unit overlapping partly the neighboring immediately adjacent stent-graft unit with the proximal stent-graft unit slip fitted or disposed partially within the inner diameter of the immediately adjacent distal stent-graft unit and overlapping the immediately adjacent distal stent-graft unit. The inner diameter of the proximal most stent-graft unit 9 is different and comparatively larger than the inner diameter of the distal most stent-graft unit 10. The multi-unit stent-graft defines a prosthesis comprising two or more stent-graft units stacked and nested with the proximal stent-graft unit overlapping partly the distal adjacent stent-graft unit. A stent-graft unit 3 is sutured together by its graft to its adjacent stent-graft unit to its graft. Stacked stent-graft units may have successively smaller outer diameters ranging from the proximal most stent-graft unit to the distal most stent-graft unit to form a general taper or conical shape longitudinally along the entire multi-unit stent-graft 1 as shown in FIG. 1. The outer diameter of the tubular multi-unit stent-graft 1 can also be the same throughout. The thickness of the graft may also vary wherein the proximal inner diameter or outer diameter and the distal inner or outer diameter are different, forming a tapered wall.

An open-frame stent 2 may be coupled to either end of the multi-unit stent-graft with one open-frame stent coupled to one end of the stent-graft or two open stents on both ends of the stent-graft. The proximal stent-graft unit 3 may be sutured outside of the distal adjoining stent-graft unit 3 or inside of the distal adjoining stent-graft unit. The overlapping length 8 between two neighboring stent-graft units 3 may be approximately 3 mm to approximately 15 mm, but preferably from approximately 5 mm to approximately 10 mm. The overlapping length 8 can vary according to the inner diameter of the stent-graft.

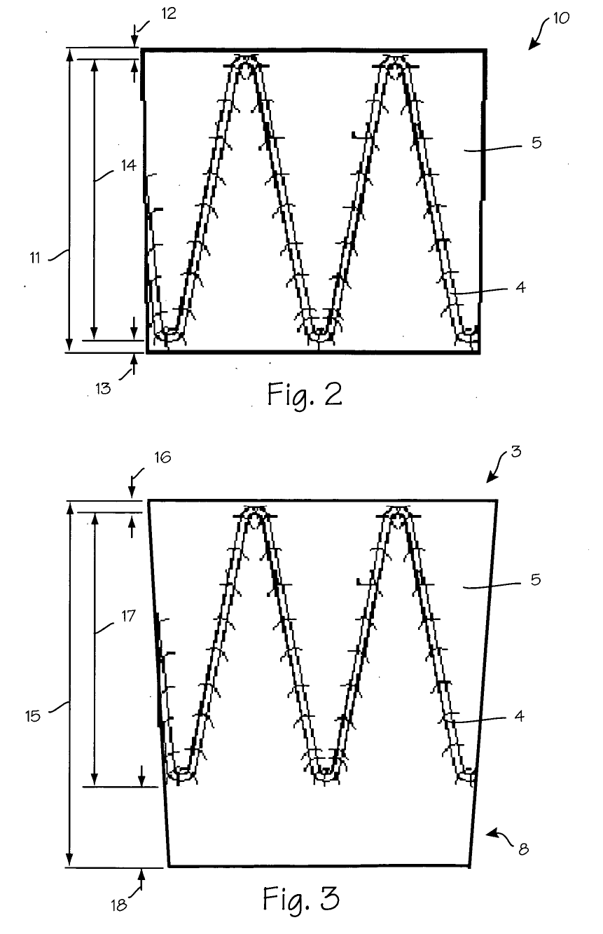

FIG. 2 and FIG. 3 illustrate stent-graft units. Each stent-graft unit 3 and 10 comprises a stent 4 sutured to a graft 5 made of biocompatible material defining a tube. The tubular grafts 5 may manufactured from polyester fabric such as DACRON®, polypropylene, polyethylene terephthalate (PET) polytetrafluoroethylene (PTFE) Or other suitable biocompatible material.

As illustrated in FIG. 2, a distal stent-graft unit 10 may comprise a graft 5 having a longitudinal length 11 with a first end portion 12 extending proximally and a second end portion 13 extending distally beyond the length of the stent 14. In this case, preferably the first 12 and second 13 end portions of the graft that extend beyond the stent are no more than approximately 3 mm in length on both end portions. As illustrated in FIG. 3, a stent-graft unit 3 may comprise a graft 5 having a length 15 with a first end portion 16 extending proximally beyond the length 17 of the stent slightly and a second end portion 18 extending distally beyond the stent by a greater length. Here, the first end portion 16 of the graft extends beyond the stent by no more than approximately 3 mm in length. The second end portion 18 extends distally beyond the stent by a greater distance creating the overlapping length. Preferably, the second end portion of the graft extending beyond the length-of the stent is no less than approximately 5 mm forming the overlapping length.

The inner diameter of the proximal most stent-graft unit 9 in a multi-unit stent-graft can be equal to the inner diameter of the distal most stent-graft unit. Alternatively, the inner diameter of the proximal most stent-graft unit 9 may be different from the inner diameter of the distal most stent-graft unit 10 in a multi-unit stent-graft. Stent-graft units in a multi-unit stent-graft 1 may be tapered or fashioned as truncated cones where the outer diameter of the second end portion of a proximal stent-graft unit is slightly smaller than the inner diameter of the first end portion of the adjoining proximal stent-graft unit. Thus, a multi-unit stent-graft 1 may comprise a series of stent-graft units 3 where the outer diameter of the second end portion of stent-graft unit is slightly smaller than the inner diameter of the first end portion of the stent-graft unit located distally to it.

The support stent forming a stent-graft unit 3 may be sutured outside of the tubular graft 5 or inside of the tubular graft 5. A support stent 4 may be formed from a single continuous wire having two ends joined together arranged in a tubular configuration with a plurality of bends having multiple undulations. The support stent 4 is in substantially sinusoidal wave shape. Each undulation in the support stent has an apex. Typically, a support stent has 3 to 10 apex or crests 19 forming a frame loop. Preferably 4 to 6 crests are in a frame loop. The number of crowns will vary according to the inner diameter of the stent-graft. The longitudinal length of a stent 4 is approximately 10 mm to approximately 40 mm, but preferably 15 mm to 25 mm. The length of a stent will vary according to the inner diameter of the stent-graft. The stent 4 may be manufactured from nitinol, stainless steel or other biocompatible alloys. Undulations in the support stent 4 of adjacent stent-graft units are generally in-phase with one another as illustrated in FIG. 1.

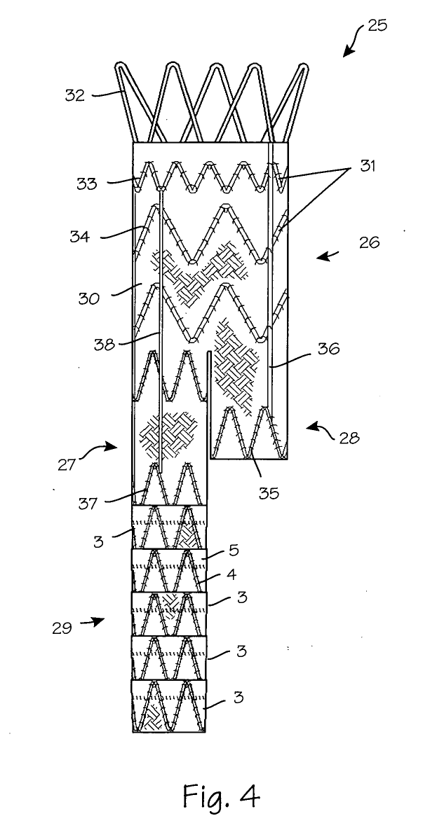

FIG. 4 illustrates a uniform bifurcated endoluminal prosthesis 25 comprising a trunk stent-graft portion 26 with a first iliac branch portion 27 and a second iliac branch portion 28 and a first iliac branch extension 29. The first iliac branch portion is in fluid communication with the second iliac branch portion. The trunk stent-graft portion comprises a trunk graft 30 with two branches on its distal end and one or more support stents 31 disposed therein. The stent 31 may be disposed within the inner diameter of the trunk graft 30, disposed about the outer diameter of the graft 30 or disposed within the matrix of the trunk graft. A stainless steel or nitinol open-frame stent 32 is coupled to the proximal section of the trunk graft 30. The open-frame stent partially overlaps the proximal end of the trunk stent-graft portion and is sutured inside. The length of the trunk graft 30 may vary according to the distance of abdominal artery between the root of renal artery and the root of the normal iliac artery. An open-frame stent 32 may be coupled to any end of the bifurcated endoluminal prosthesis including the proximal end of the trunk stent-graft or the distal ends of the first iliac branch portion or the second iliac branch portion. Thus, the bifurcated endoluminal prosthesis may have one, two or three open-frame stents or none at all.

Disposed distal to the open-frame stent 32 is a stent in the form of a small sine wave or mini-wave stent 33 sutured inside of the proximal section of the trunk graft. Disposed distally to the mini-wave stent 33 are two larger-sized stent 34 in the form of a sine wave sutured inside of the proximal end portion of the trunk graft portion. One or more larger-sized stents 34 adjacent to the mini-wave stent may be sutured to the proximal section of the trunk graft portion 26. The quantity of stent disposed within the trunk stent-graft portion may vary according to the length of the trunk.

The mini-wave stent 33 is a smaller sized stent comprising a thinner gauge wire formed in a substantially sinusoidal wave shape having a shorter wave height than that of the larger-sized support stents 4. The mini-wave stent typically has a shorter wavelength resulting in more apex or crests in a loop than the larger-sized support stents 4. The mini-wave stent enforces the inosculation between the graft and blood vessel and helps to diminish the proximal endo-leak of the stent-graft. Use of a mini-wave stent assists the stent-graft in treatment of aneurysms having a shorter neck. The mini-wave stent 33 maintains stent supporting force which ensures stent-graft fixation to the graft and prevents stent-graft migration.

The first iliac branch portion 27 and the second iliac branch portion 28 comprise one or more branch supporting stents sutured inside or outside. The distal most branch supporting stent 35 of one branch portion connects to the proximal open-frame stent with a first longitudinal bar 36. The distal most branch supporting stent 37 of the other branch is coupled to the mini-wave stent using a second longitudinal bar 38. Alternatively, the distal most stent branch supporting stent may be coupled to the first larger-sized stent 34 adjacent and distal to the mini-wave stent with a longitudinal bar.

A branch supporting stent 35 is disposed within and sutured to the distal portion of the second iliac branch portion. A longitudinal bar 36 disposed within the trunk stent-graft portion extends from the open-frame stent to the stent 35 disposed on the distal section of the second iliac branch portion. The first iliac branch portion comprises two branch supporting stents sutured to the inside of the trunk graft. One stent is located on the proximal section and the other is located on the distal section of the first iliac branch portion. A second longitudinal bar 38 extends from the distal most stent 37 of the other branch to the mini-wave stent 33.

A first iliac branch extension 29 is sutured to the first iliac branch portion. An overlapping length on the first iliac branch portion extends beyond the stent and partially overlaps the distal end of the first iliac branch extension. The first iliac branch extension may comprise a multi-unit stent-graft as illustrated in FIG. 1. The first iliac branch extension comprises two or more stent-graft units stacked upon each other and inter-connected. Each stent-graft unit 3 comprises a stainless steel or nitinol stent 4 sutured to a graft 5 made of biocompatible PET woven fabric defining a tube or having an inner diameter, outer diameter and a length in the longitudinal direction. The stent may be disposed within the inner diameter of the graft, disposed about the outer diameter of the graft or disposed within the matrix of the PET woven fabric. The supporting stent 4 forming the stent-graft unit 3 may be sutured inside or outside of the graft 5 made of biocompatible material defining a tube. A distal most stent-graft unit may comprise a graft with a first end portion and a second end portion slightly exceeding the length of the stent. Other stent-graft units 3 may have a graft with the first end portion slightly exceeding the length of the stent and the second end portion exceeding the length of the stent to a greater extent creating an overlapping length in the stent-graft unit.

The stent-graft units 3 are stacked, nested, interconnected and sutured together with the proximal stent-graft unit disposed partially within the inner diameter of the immediately adjacent distal stent-graft unit and overlapping the immediately adjacent distal stent-graft unit. The proximal stent-graft unit may be sutured inside or outside of the neighboring distal stent-graft unit. The inner and outer diameter of the first iliac branch extension can be the same throughout or vary wherein the proximal inner or outer diameter and the distal inner or outer diameter are different forming a taper. The first iliac branch extension may also be provided with an open stent sutured on the distal end.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

1. A tubular endoluminal prosthesis comprising:
a plurality of stent-graft units with a proximal stent-graft unit overlapping partly and coupled to an immediate adjacent distal stent-graft unit, said stent-graft units comprising a supporting stent coupled to a tubular graft.

2. The tubular endoluminal prosthesis of claim 1 wherein each stent-graft is coupled to its immediate adjacent distal stent-graft by suturing the graft of the proximal stent-graft to the graft of the immediate adjacent distal stent graft.

3. The tubular endoluminal prosthesis of claim 1 further comprising an open-frame stent overlapping partly and coupled to the proximal most stent-graft unit.

4. The tubular endoluminal prosthesis of claim 1 further comprising an open-frame stent overlapping partly and coupled to the distal most stent-graft unit.

5. The tubular endoluminal prosthesis of claim 3 further comprising an open-frame stent overlapping partly and coupled to the distal most stent-graft unit.

6. The tubular endoluminal prosthesis of claim 1 wherein the graft comprises a material selected from the group consisting of polyester fabric, polypropylene, polyethylene terephthalate (PET) and polytetrafluoroethylene (PTFE).

7. The tubular endoluminal prosthesis of claim 1 wherein the stent comprises nitinol or stainless steel.

8. The tubular endoluminal prosthesis of claim 1 wherein at least one stent-graft unit comprises a graft with a first end and a second end slightly exceeding the longitudinal length of the stent.

9. The tubular endoluminal prosthesis of claim 1 wherein at least one stent-graft unit comprises a graft with a first end slightly exceeding the longitudinal length of the stent and a second end exceeding the longitudinal length of the stent by a greater length than the first end.

10. The tubular endoluminal prosthesis of claim 1 wherein the supporting stent is formed from a single continuous wire arranged in a tubular configuration with multiple bends and having multiple undulations with each undulation having an apex.

11. The tubular endoluminal prosthesis of claim 10 wherein the undulations in adjacent stent-graft units are generally in-phase with one another.

12. The tubular endoluminal prosthesis of claim 1 wherein a distal most stent-graft unit has a different outer diameter from that of the proximal most stent-graft unit.

13. A bifurcated endoluminal prosthesis comprising:
a trunk stent-graft portion having a first iliac branch portion and a second iliac branch portion, said trunk stent-graft portion comprising:
a trunk graft made of biocompatible material defining a tube with two branches on the distal end; and p1 a mini-wave stent sutured inside a proximal section of the trunk graft;
one or more supporting stents disposed adjacent to and distal to the mini-wave stent within the proximal section of the trunk graft and
one or more branch supporting stent sutured to the first iliac branch portion of the trunk graft with the distal most branch supporting stent being connected to the mini-wave stent using a first bar.

14. The bifurcated endoluminal prosthesis of claim 13 wherein the trunk graft comprises a material selected from the group consisting of polyester fabric, polypropylene, polyethylene terephthalate (PET) and polytetrafluoroethylene (PTFE).

15. The tubular endoluminal prosthesis of claim 13 wherein the supporting stent comprises nitinol or stainless steel.

16. The bifurcated endoluminal prosthesis of claim 13 further comprising an open-frame stent coupled to and overlapping partly a proximal end of the trunk graft.

17. The bifurcated endoluminal prosthesis of claim 16 further comprising one or more branch supporting stents sutured to the second iliac branch portion with the distal most branch supporting stent in the second iliac branch portion connected to the open-frame stent using a second bar.

18. The bifurcated endoluminal prosthesis of claim 13 further comprising a first iliac branch extension coupled to the first iliac branch portion, said first iliac branch extending comprising a plurality of stent-graft units with a proximal stent-graft unit overlapping partly and coupled to the immediate adjacent distal stent-graft unit, said stent-graft units comprising a supporting stent coupled to a tubular graft.

19. The bifurcated endoluminal prosthesis of claim 18 wherein the stent graft units are coupled by suturing the graft of the proximal stent-graft unit to the graft of the immediate adjacent distal stent-graft unit.

20. The bifurcated endoluminal prosthesis of claim 13 wherein the first iliac branch extension further comprises an open-frame stent overlapping partly and coupled to the distal most stent-graft unit.

21. The bifurcated endoluminal prosthesis of claim 18 wherein the first iliac branch extension further comprises at least one stent-graft unit having a graft with a first end slightly exceeding the longitudinal length of the stent and a second end exceeding the longitudinal length of the stent by a greater length than the first end.

22. The bifurcated endoluminal prosthesis of claim 18 wherein the supporting stent is formed from a single continuous wire arranged in a tubular configuration with multiple bends and having multiple undulations with each undulation having an apex.

23. The bifurcated endoluminal prosthesis of claim 22 wherein the undulations in adjacent stent-graft units are generally in-phase with one another.

24. The bifurcated endoluminal prosthesis of claim a distal most stent-graft unit has a different outer diameter m that of the proximal most stent-graft unit.
